(19) United States
(12) Patent Application Publication
(10) Pub. No.: US 2010/0076348 A1
(43) Pub. Date: Mar. 25, 2010

(54) COMPLETE INTEGRATED SYSTEM FOR CONTINUOUS MONITORING AND ANALYSIS OF MOVEMENT DISORDERS

(75) Inventors: James McNames, Portland, OR (US); Pedro Mateo Riobo Aboy, Beaverton, OR (US); Andrew Greenberg, Portland, OR (US)

Correspondence Address: ABOY&ASSOCIATES PC;www.aboypatentlaw.com, 522 SW 5th Ave, Suite 1265, Portland, OR, 97204, US

(73) Assignee: APDM, INC, Portland, OR (US)

(21) Appl. No.: 12/565,697

(22) Filed: Sep. 23, 2009

Related U.S. Application Data
(60) Provisional application No. 61/099,204, filed on Sep. 23, 2008.

(51) Int. Cl. A61B 5/11

(52) U.S. Cl. 600/595

(57) ABSTRACT

Disclosed embodiments include a complete integrated system designed to support continuous monitoring and objective analysis of movement disorders. According to one embodiment the integrated system allows for continuous monitoring of movement disorders during normal daily activities in home and other normal daily environments, as well as in the clinic. The integrated system comprises: 1) wearable movement monitoring devices including a plurality of inertial sensors, 2) a docking station with wireless capabilities, 3) a secure web-enabled data server, and 4) statistical signal processing methods, all of which are integrated to enable monitoring and analysis of movement disorders.

### CROSS-REFERENCE TO RELATED APPLICATIONS

This application claims the benefit of U.S. Provisional Application No. 61/099,204 filed on 2008 Sep. 23 by the present inventors, which is incorporated herein by reference.

### BACKGROUND

1. Field of Invention

This invention is related to systems for supporting clinical research and clinical practice. Specifically, this invention relates to systems especially adapted for movement disorders.

2. Related Art

Parkinson's disease (PD) is the second most common neurodegenerative disease and the most common serious movement disorder. It afflicts approximately 1 million in the US alone costing the economy over $25 billion annually. Levodopa is the most potent antiparkinson drug and is the primary therapy for most patients. However, continual use of levodopa over time causes fluctuations in bradykinesia (slowness of movement), tremor, and dyskinesia (uncoordinated writhing movements) and has variable effects on gait and posture. Accurate assessment of Parkinsonian motor impairments is crucial for optimizing therapy in clinical practice and for determining efficacy of new therapies in clinical trials. Subjective clinical rating scales such as the Unified Parkinson's Disease Rating Scale (UPDRS) are the most widely accepted standard for motor assessment. Objective static devices have also been developed to assess impairment more accurately and consistently. However, the value of both subjective and objective forms of static motor assessment may be limited in certain situations because each patient's motor state varies continuously throughout the day.

In recent years, large advances have been made in micro-electro-mechanical systems (MEMS) and inertial sensors, in particular. It is now possible to record body movements with devices that include accelerometers, gyroscopes, goniometers, and magnetometers. However, the feasibility of using these sensors to quantify motor deficits associated with PD remains unknown.

Current inertial monitoring systems can be divided into three categories: computer-tethered, unit-tethered, and untethered. Computer-tethered devices connect the sensor directly to a computer using a wireline connection. Unit-tethered systems connect the sensors to a central recording unit using a wireline connection that is typically worn around the waist.

The only wireless untethered systems reported in the literature are “activity monitors,” which measure the coarse degree of activity at intervals of 1-60 s, typically with a wrist-worn device that contains a single-axis accelerometer. These devices are sometimes called actigraphs or actigraphers. Their low sampling frequency makes them inadequate for most movement disorder applications.

Most prior work on continuous monitoring of PD has used unit-tethered systems during in-patient studies. Most of these studies have used accelerometers and some have used gyroscopes.

Currently there are no systems or detailed automatic methods designed to obtain impairment indices for movement disorders such as Parkinson's disease or essential tremor in continuous monitoring settings in order to help guide therapy and/or continuously monitor the symptoms of movement disorders. Specifically, there are no solutions currently available that include a complete integrated system to perform collection, monitoring, uploading, analysis, and reporting of movement data.

### SUMMARY

Disclosed embodiments include a complete integrated system designed to support continuous monitoring and objective analysis of movement disorders. For example, and without limitation, the integrated system is especially adapted for movement disorders such as Parkinson's disease. The most basic embodiment includes a complete integrated system which allows for continuous monitoring of movement disorders during normal daily activities in home and other normal daily environments, as well as in the clinic; comprising: 1) wearable movement monitoring devices, 2) a docking station, 3) a data server, and 4) statistical signal processing methods, all of which are integrated to enable monitoring and analysis of movement disorders.

### BRIEF DESCRIPTION OF THE DRAWINGS

Disclosed embodiments are illustrated by way of example, and not by way of limitation, in the figures of the accompanying drawings.

FIG. 1 illustrates a block diagram of the system according to one embodiment.

FIG. 2 illustrates a block diagram of a web-enabled data server according to one embodiment.

FIG. 3 illustrates an embodiment of a wearable device for movement monitoring.

FIG. 4 illustrates an embodiment of a docking station.

FIG. 5 illustrates an embodiment of a docking station.

FIG. 6 illustrates an embodiment of a docking station.

FIG. 7 illustrates an embodiment of a docking station with a wearable movement monitoring device docketed.

FIG. 8 shows a block diagram of the integrated systems components according to one embodiment.

### DETAILED DESCRIPTION

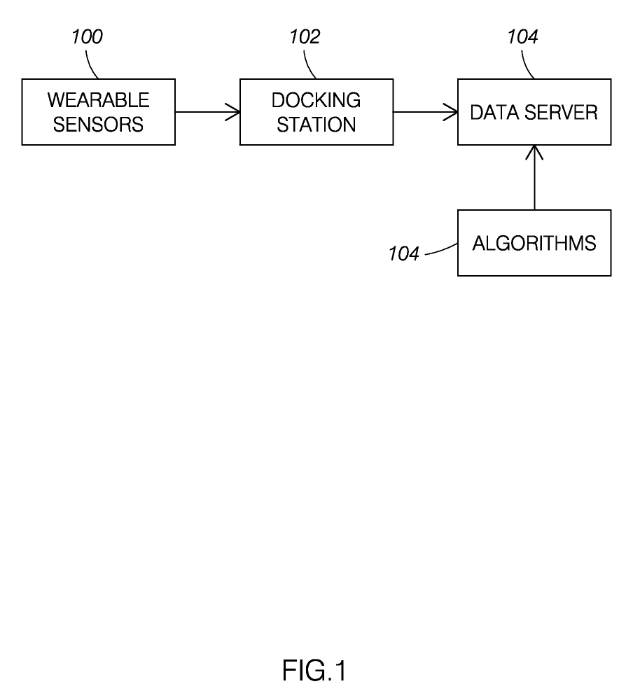

FIG. 1 illustrates a block diagram of the system according to one embodiment. In one embodiment, the integrated system comprises: wearable devices 100, a docking station 102, a data server 104, and analysis algorithms 106.

According to one embodiment, the wearable devices 100 are compact devices that continuously record data from embedded sensors. The sensors 100 may be worn at any convenient location on the body that can monitor impaired movement. Convenient locations include the wrists, ankles, waist, sternum, pocket, upper arms, and thighs. In one embodiment, the sensors include one or more channels of electromyography, accelerometers, gyroscopes, magnetometers, or other small sensors that can be used to monitor movement. The wearable sensors 100 have sufficient memory and battery life to continuously record inertial data throughout the day from the moment subjects wake up until they go to sleep at night, typically 18 hours or more (in a particular embodiment the wearable devices include sufficient storage to log data for several weeks). The sensors 100 automatically start recording when they are removed from the docking station. In one embodiment, there is no need for the user to turn them on or off.

According to one embodiment, and without limitation, in order to facilitate use in the home and other normal daily environments, the system includes a docking station 102 that is used to charge the batteries of the wearable devices 100 and download the data from each day of activities. The docking station 102 uploads the data using whatever means are available in that setting. If high-speed Internet access is available within the home, this may be used for data upload. Alternatively, it permits the user to download the data to a portable storage device such as a USB thumb drive or hard drive that can then be transported to a site for final upload to the data server. If there is no simple means to download the data from the docking station 102, the data is downloaded once the docking station is returned at the end of the monitoring period. The docking station 102 requires no user intervention. The devices 100 stop recording as soon as they are docked and start recording as soon as they are undocked. According to one embodiment, the docking station 102 does not include any buttons. The docking station 102 can be connected to a computer for data extraction and processing, but this is optional. Several docking stations 102 can be connected together to charge a plurality of wearable movement devices 100. Movement data can be transmitted wirelessly from a plurality of wearable movement devices 100 to the docking station 102 or directly to the data server 104.

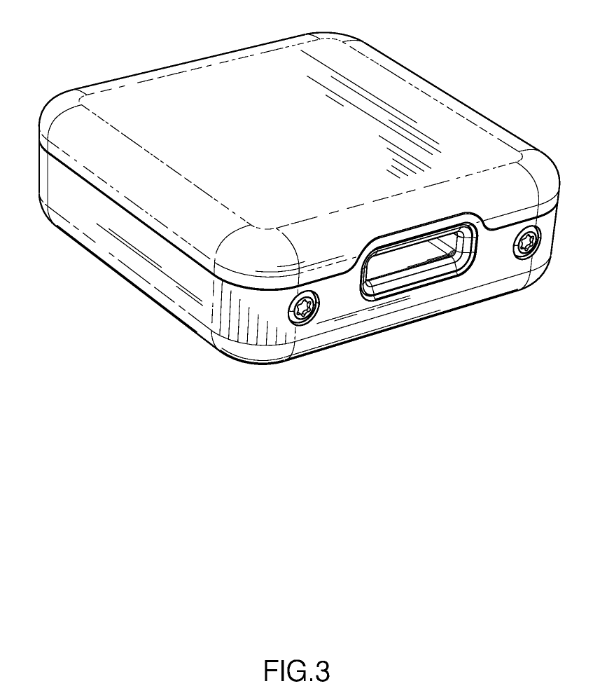

FIG. 3 illustrates an embodiment of a wearable device for movement monitoring. The wearable movement devices 100 comprise: (a) a power source, (b) a local storage memory, (c) a microcontroller, (d) a wireless transmitter circuit for wirelessly transmitting said plurality of movement data to a wireless receiver, and (e) a plurality of movement sensors including 3-axis accelerometers, gyroscopes, and magnetometers.

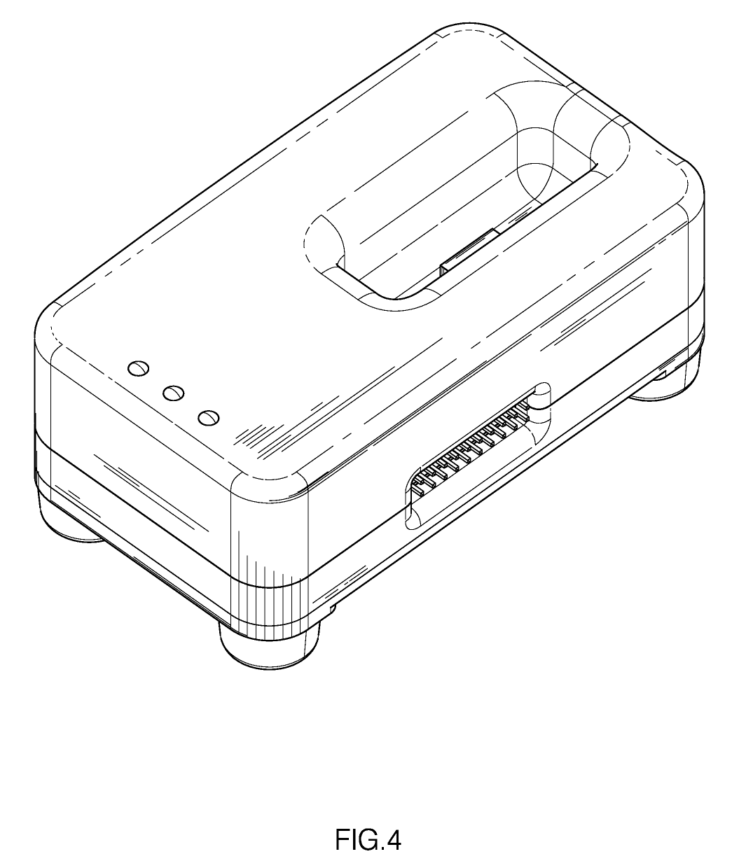

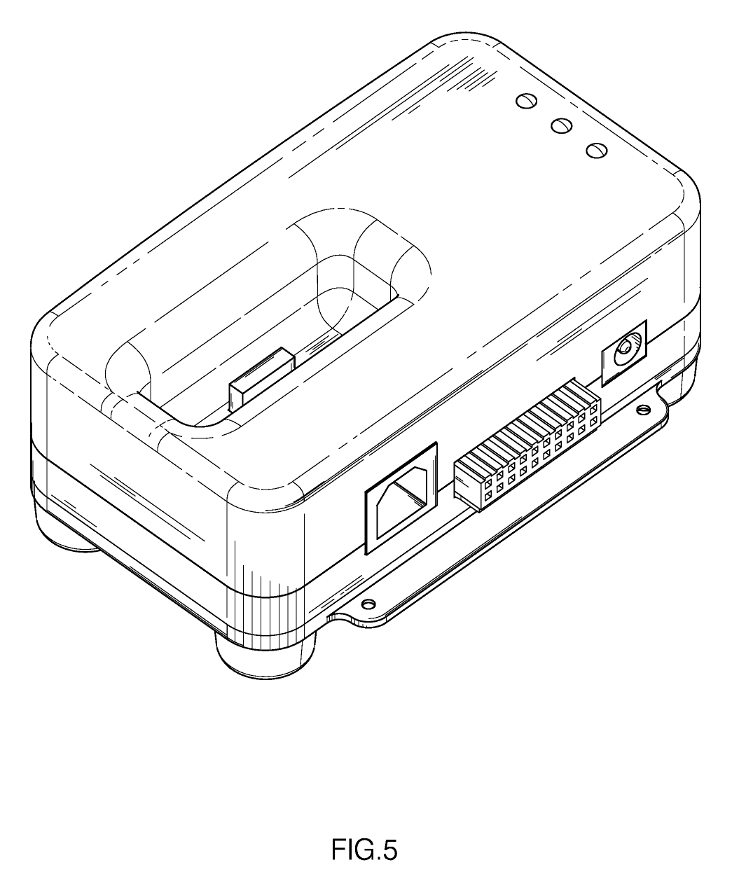

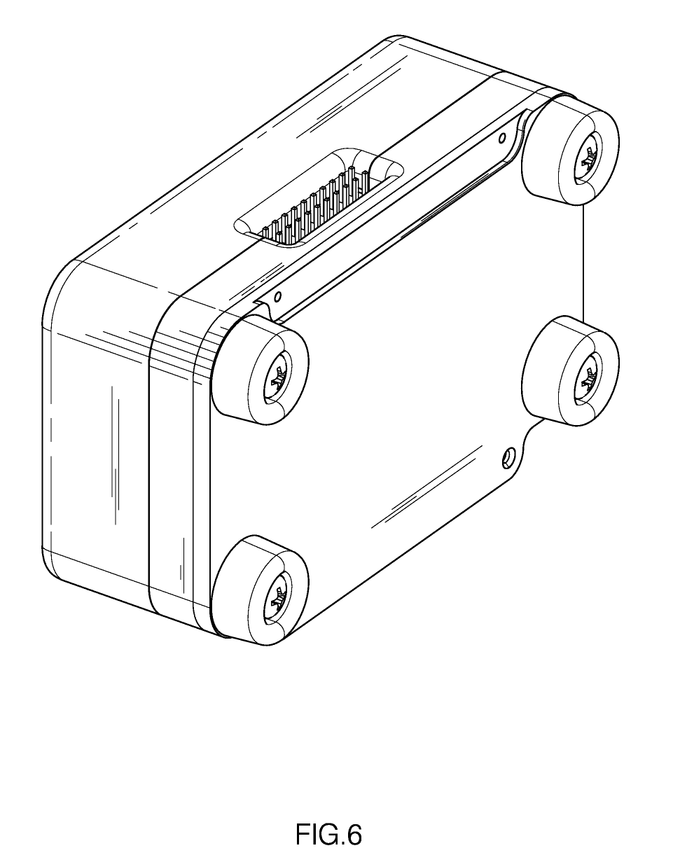

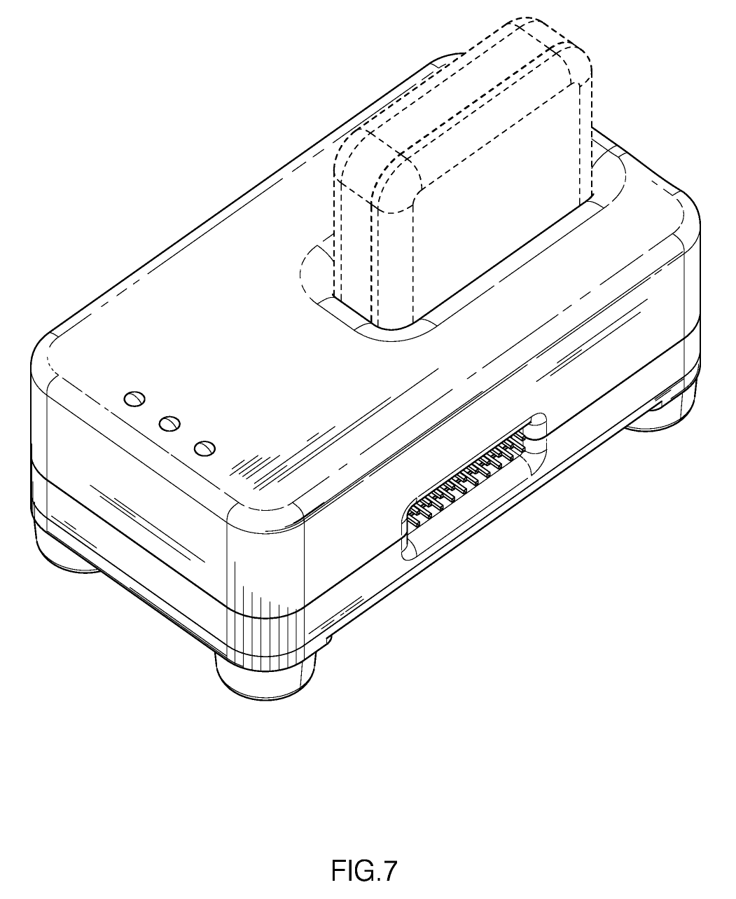

FIG. 4, FIG. 5 and FIG. 6 illustrate an embodiment of a docking station. According to an embodiment, the integrated movement monitoring system includes one or more docking stations, said docking station comprising: (a) a power supply for powering said docking station 102, (b) one or more charging dockets for re-charging said one or more wearable movement monitoring devices, and (c) an integrated base station. According to a particular embodiment, the integrated based station comprises: (a) a power source, (b) a wireless receiver circuit; (c) a wireless transmitter circuit, and (d) one or more connections to a digital computer. FIG. 7 illustrates an embodiment of a docking station with a wearable movement monitoring device docketed.

Once the data is uploaded to the server 104, the server 104 runs automatic algorithms (digital signal processing methods) 106 to analyze the data and compute the results needed for the application. The system provides data for three applications: 1) human movement research, 2) movement disorders studies and clinical trials, and 3) clinical care. The system provides a simple means for researchers to conduct studies in human movement with wearable sensors 100. Study participants have an easy means of handling the devices by simply docking them when not in use. Researchers have easy, secure, and protected access to their raw sensor data through the server 104. The system also provides full support for research studies and clinical trials in movement disorders such as Parkinson's disease and essential tremor. It permits researchers to easily upload other types of data such as clinical rating scale scores, participant information, and other types of device data integrated into a secure database, and provides a means for sharing the data. Different views and controlled access permit study coordinators, research sponsors, statisticians, algorithm developers, and investigators to easily monitor the progress of studies and results. The system also provides the ability to do sequential analysis for continuous monitoring of clinical studies. The system has strict, secure, and encrypted access to any protected health information that is stored in the server. The system also supports clinical monitoring of individual patients to determine their response to therapy. This is especially helpful for movement disorders such as advanced Parkinson's in which the degree of motor impairment fluctuates continously throughout the day. As with clinical studies and trials, the server provides secure, encrypted access to patient records for authenticated care providers as well as patients themselves.

According to one embodiment, the algorithms 106 process the raw device data and extract the metrics of interest. These algorithms are insensitive to normal voluntary activities, but provide sensitive measures of the motor impairments of interest. In Parkinson's disease this may include tremor, gait, balance, dyskinesia, bradykinesia, rigidity, and overall motor state.

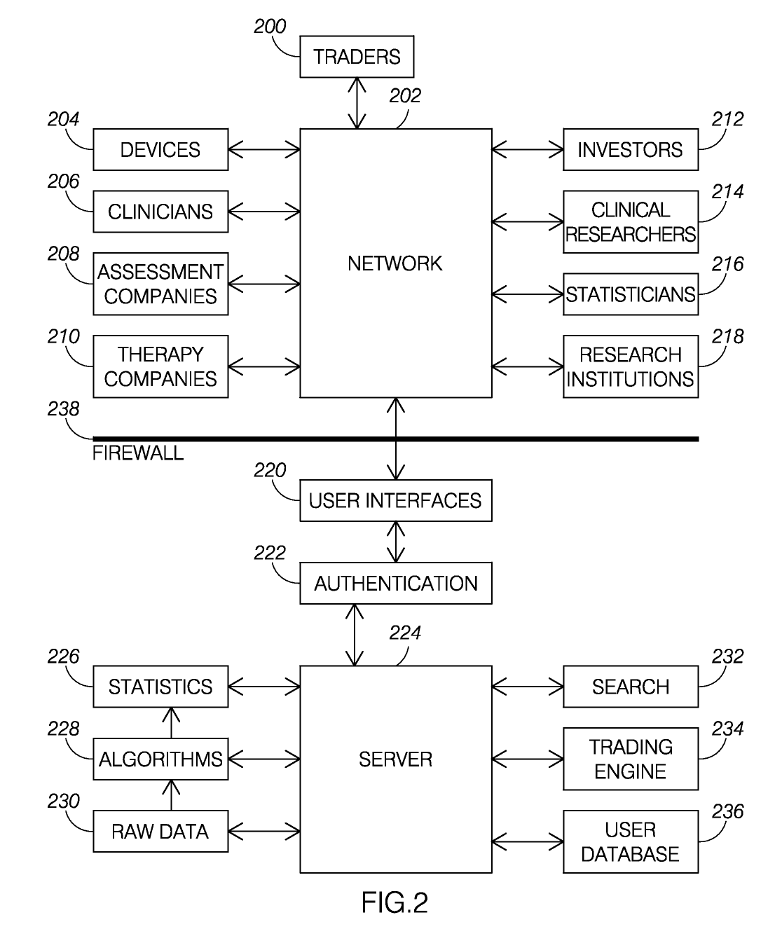

FIG. 2 illustrates a block diagram of a web-enabled data server according to one embodiment. It illustrates an example of a system architecture according to one embodiment of the invention where the platform serves to enable collaboration among the different stakeholders involved in research. In this embodiment, traders 200, devices 204, clinicians 206, assessment companies 208, therapy companies 210, investors 212, clinical researchers 214, statisticians 216, and research institutions 218 are connected to a network 202 with access to a central server 224 through a secured firewall 238. Each user goes through a user-specific authentication procedure 222 and has a user-specific interface 220. According to this embodiment the system components comprise a central server 224, a database to store raw data 230, algorithms 228 to analyze raw data and create user specific reports, a user database 236, a statistics module 226, a trading engine 234, and search capabilities 232.

In one embodiment the system includes a web server 104 that runs an integrated online platform designed for mass collaboration. It supports encrypted data transfer through standard encryption protocols. A relational database such as MySQL is used to store user profiles, protocols, study data, study results, and collaboration team information. The system is built using standard server practices with the best practices of security, backups, and redundancy. All users are authenticated and the data is carefully controlled to ensure compliance with federal regulatory requirements such as the Health Information Portability and Accountability Act (HIPAA).

According to one embodiment, the system includes functionality to enable researchers to conduct prospective trials in which the hypotheses are stated prior to any data collection and the statistical analysis is automated and finalized prior the study initiation (i.e. locked down). This prevents researchers from trying other analysis methodologies during the course of their study until they find one that is favorable, which leads to a higher prevalence of false positives than expected.

According to another embodiment, the system includes functionality to enable analysts and researchers to perform an exploratory analysis of the data as it arrives. This embodiment is designed to facilitate faster identification of new metrics and provide the rest of the community with faster information about whether new therapies look promising or not.

Another embodiment of the system includes functionality to enable the research community to conduct larger meta studies with the raw data. Typically, a meta analysis, which pools the data together from multiple studies, can only be applied to the published results. The system permits the meta analysis to be performed on the raw data, which leads to more statistical power and faster discovery of new knowledge.

Another embodiment combines each of the embodiments described above into a single integrated collaboration platform which includes functionality to enable data sharing, data analysis, knowledge creation and sharing, problem solving, and accelerated scientific discovery by collaborating teams which may be formed on an ad-hoc basis among users of the system. The platform is designed to accelerate research and improve clinical care of chronic conditions. It provides a central place to facilitate interactions between the many different groups that participate in these activities. The central features of the system can be tailored to best suit each chronic condition. In this embodiment, the system brings clinical researchers, engineers, scientists, medical doctors, patients, family, pharmaceutical companies, statisticians, research institutions, investors, and traders together in one “place” (integrated collaboration platform system) and promotes community and collaboration on chronic conditions. In this embodiment, data may be open and anyone can download it or access it. The system may include sunrise dates for new data after which the data becomes open to the public. Additionally, automatic data analysis is conducted using state of the art biomedical signal processing algorithms and reports are generated. As a marketplace, investors may help fund studies, drug trials, new technologies, and other improvements in therapies. Patients, researchers, clinicians, and collaborators can suggest and design trials for new therapies.

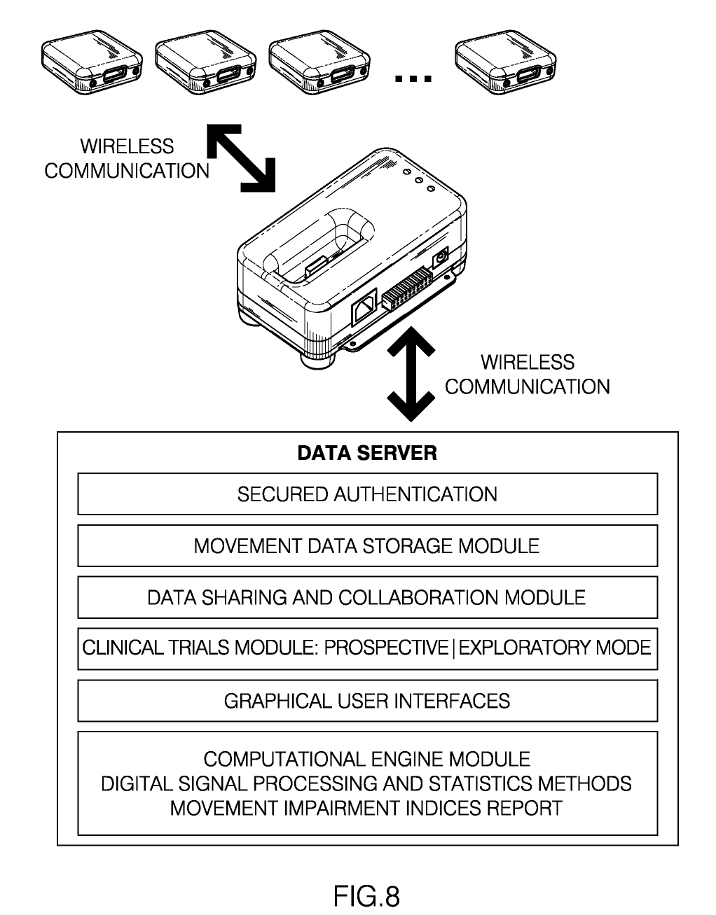

FIG. 8 shows a block diagram of the integrated systems components according to one embodiment. In this embodiment, a plurality of wearable movement monitors 100 collect a plurality of movement data and wirelessly transmit synchronized movement data collected in a plurality of locations to one or more docking stations 102 that include a base station with wireless transceiver and storage capabilities. The wearable movement monitors 100 or the docking stations 102 wireless transmit said plurality of movement data to a secure data server that includes a clinical data management system especially adapted for movement disorders (substantially equivalent embodiments include data transmitted through any means of Internet access, such as DSL, cable modems, or dedicated access). The secure data server 104 is a web-enabled clinical data management system especially adapted for (a) storing, (b) sharing, (c) managing, and (d) analyzing movement disorder data. The web-enabled clinical data management comprises: (a) a secure data storage module, (b) a secure data sharing and collaboration module, (c) a secure data management module, (d) a computational engine module, and (e) a plurality of graphical user interfaces; whereby said computational engine module comprises one or more digital signal processing and statistics methods 106 for analysis and processing of said plurality of movement disorder data and automatically generating a report comprising (a) plurality of movement impairment indices and (b) a plurality of clinical scores such as a tremor index, a dyskinesia index, and a bradykinesia index; as well as gait, balance, overall motor state indices, multiple sclerosis, stroke, and other neurological injuries and disorders that lead to impaired movement such as traumatic brain injury. According to this embodiment, said report is a downloadable report including a plurality of results including a plurality of (a) numerical results, (b) summary statistical results, (c) tables, (d) time domain plots, (e) frequency domain plots, and (f) time-frequency plots such as spectrograms.

According to one embodiment, the integrated system described above is focused on Parkinson's disease. In another embodiment the system is focused on essential tremor. In another embodiment the system is focused on general movement disorders.

While particular embodiments and example results have been described, it is understood that, after learning the teachings contained in this disclosure, modifications and generalizations will be apparent to those skilled in the art without departing from the spirit of the disclosed embodiments.

The invention claimed is:

1. An integrated movement monitoring system, comprising:
(a) one or more wearable movement monitoring devices comprising one or more movement sensors for collecting a plurality of movement data; and
(b) at least one secure data server for storing said plurality of movement data, said secure data server implemented in a digital computer with one or more processors.

2. The integrated movement monitoring system of claim 1, whereby said one or more movement sensors comprise one or more inertial sensors.

3. The integrated movement monitoring system of claim 2, whereby said inertial sensors include one or more accelerometers.

4. The integrated movement monitoring system of claim 3, whereby said one or more accelerometers are 3-axis accelerometers.

5. The integrated movement monitoring system of claim 4, whereby said inertial sensors include one or more gyroscopes.

6. The integrated movement monitoring system of claim 5, whereby said one or more movement sensors include one or more magnetometers.

7. The integrated movement monitoring system of claim 6, whereby said one or more wearable movement monitoring devices are wireless devices further comprising:
(a) a power source;
(b) a local storage memory;
(c) a microcontroller, and
(d) a wireless transmitter circuit for wireless transmitting said plurality of movement data to a wireless receiver.

8. The integrated movement monitoring system of claim 7, further comprising one or more docking stations, said docking station comprising:
(a) a power supply for powering said docking station;
(b) one or more charging dockets for re-charging said one or more wearable movement monitoring devices; and
(c) an integrated base station.

9. The integrated movement monitoring system of claim 8, whereby said integrated base station comprises:
(a) a power source;
(b) a wireless receiver circuit; and
(c) a wireless transmitter circuit.

10. The integrated movement monitoring system of claim 9, whereby said integrated based station further comprises a connection to a digital computer.

11. The integrated movement monitoring system of claim 10, whereby said digital computer is a regulatory compliant compliant secure data server.

12. The integrated movement monitoring system of claim 11, whereby said secure data server is a web-enabled clinical data management system especially adapted for (a) storing, (b) sharing, (c) managing, and (d) analyzing movement disorder data.

13. The integrated movement monitoring system of claim 12, whereby said web-enabled clinical data management system comprises:
(a) a secure data storage module;
(b) a secure data sharing and collaboration module;
(c) a secure data management module;
(d) a computational engine module; and
(e) a plurality of graphical user interfaces.

14. The integrated movement monitoring system of claim 13, whereby said computational engine module comprises one or more digital signal processing and statistics methods for analysis and processing of said plurality of movement disorder data and automatically generating a report comprising (a) a plurality of movement impairment indices and (b) a plurality of clinical scores.

15. The integrated movement monitoring system of claim 14, whereby said plurality of movement impairment indices comprise (a) a tremor index, (b) a dyskinesia index, and (c) a bradykinesia index.

16. The integrated movement monitoring system of claim 15, whereby said plurality of movement impairment indices further comprises gait, balance, and overall motor state indices, multiple sclerosis, stroke, and neurological injuries and disorders that lead to impaired movement such as traumatic brain injury.

17. The integrated movement monitoring system of claim 16, whereby said report is a downloadable report including a plurality of results including a plurality of (a) numerical results, (b) summary statistical results, (c) tables, (d) time domain plots, (e) frequency domain plots, and (f) time-frequency plots such as spectrograms.

18. The integrated movement monitoring system of claim 17, whereby said web-enabled clinical data management system includes a clinical trials module.

19. The integrated movement monitoring system of claim 18, whereby said clinical trials module further includes a prospective trials module.

20. The integrated movement monitoring system of claim 19, whereby said clinical trials module further includes an exploratory analysis module and a meta studies module.
